(19) United States
(12) Patent Application Publication
(10) Pub. No.: US 2025/0085425 A1
(43) Pub. Date: Mar. 13, 2025

(54) ALIGNMENT SYSTEMS AND METHODS

(71) Applicant: Wright Medical Technology, Inc., Memphis, TN (US)

(72) Inventors: Julia C. ALSPAUGH, Memphis, TN (US); David G. REYNOLDS, Fairport, NY (US)

(73) Assignee: Wright Medical Technology, Inc., Memphis, TN (US)

(21) Appl. No.: 18/960,387

(22) Filed: Nov. 26, 2024

Related U.S. Application Data
(63) Continuation of application No. 17/661,196, filed on Apr. 28, 2022, now Pat. No. 12,196,856.
(60) Provisional application No. 63/208,572, filed on Jun. 9, 2021.

(51) Int. Cl. G01S 17/46; A61B 6/12; A61B 17/00; A61B 90/00; G01S 5/16

(52) U.S. Cl. CPC G01S 17/46; A61B 6/12; G01S 5/166; A61B 2017/00725; A61B 2090/3966

(57) ABSTRACT

Alignment systems and methods are disclosed. A system includes a first component and a second component. The first component has a first body supporting a first alignment member. The second component has a second body supporting a second alignment member. The first and second alignment members are separated from another and are configured to provide an indication that a fluoroscopic device is properly aligned with an anatomical plane when viewed under fluoroscopy. A method includes placing a first component supporting a first alignment member and a second component supporting a second alignment member relative to a patent, and aligning a fluoroscopic device with an anatomical plane using the first and second alignment members.

### CROSS-REFERENCE TO RELATED APPLICATION

This application is a continuation of U.S. application Ser. No. 17/661,196, filed on Apr. 28, 2022, which claims priority under 35 U.S.C. § 119(e) to U.S. Provisional Application No. 63/208,572, filed Jun. 9, 2021, the entirety of which is incorporated by reference herein.

### FIELD OF DISCLOSURE

The disclosed systems and methods relate to the field of medical radiography. More particularly, the disclosed systems and methods relate to providing enhances visualization for ensuring that a prosthesis, fixture, and/or jig used in a surgical procedure is positioned properly.

### BACKGROUND

Numerous surgical procedures use fluoroscopy to confirm a surgical plan and/or a placement of a surgical device, such as a fixture, prosthesis, and/or jig relative to a patient's anatomy. Ensuring proper alignment between a fluoroscopic device (e.g., an X-ray device supported by a C-arm) and an anatomical plane and/or surgical instrument is critical to ensuring that the information provided by fluoroscopy is accurate.

### SUMMARY

In some embodiments, a system includes a first component and a second component. The first component has a first body supporting a first alignment member. The second component has a second body supporting a second alignment member. The first and second alignment members are separated from another and are configured to provide an indication that a fluoroscopic device is properly aligned with an anatomical plane when viewed under fluoroscopy.

In some embodiments, a method includes placing a first component supporting a first alignment member and a second component supporting a second alignment member relative to a patent, and aligning a fluoroscopic device with an anatomical plane using the first and second alignment members.

In some embodiments, a system includes an alignment device having a body defining a first aperture and a second aperture. The first and second apertures are disposed at an angle with one another. The first and second apertures are in communication with one another such that light passes through the first and second apertures when a fluoroscopic device is aligned with an anatomical plane.

In some embodiments, a method includes receiving light emitted from a light source in a first aperture defined by an alignment device, and directing the light from the first aperture defined by the alignment device to a second aperture defined by the alignment device such that light exits the alignment device from the second aperture. The second aperture is disposed at an angle with respect to the first aperture. The second aperture is disposed at an angle with respect to the first aperture.

In some embodiments, a system includes a first surgical device and an alignment adapted. The first surgical device is configured to be coupled to a patient. The alignment adapter has a body including a planar surface and a coupling mechanism for coupling the alignment adapter to the first surgical device such that the planar surface is parallel to an anatomical plane when the first surgical device is coupled to the patient and the alignment adapter is coupled to the first surgical device.

In some embodiments, a method includes placing a first surgical device relative to a patient, coupling an alignment device to the first surgical device, and aligning a fluoroscopic device with the planar surface of the alignment device. The alignment device includes a planar surface.

### BRIEF DESCRIPTION OF THE DRAWINGS

The description of the exemplary embodiments disclosed herein are intended to be read in connection with the accompanying drawings, which are to be considered part of the entire written description. All drawing figures are schematic illustrations and are not intended to show actual dimensions or proportions.

FIG. 1A is a first side view of first and second components each having a respective alignment member in accordance with some embodiments;

FIG. 1B is a representation of the first and second components illustrated in FIG. 1A being viewed under fluoroscopy when there is a misalignment between the fluoroscopic device and the first and second components in accordance with some embodiments;

FIG. 1C is a representation of the first and second components illustrated in FIG. 1A being viewed under fluoroscopy when the fluoroscopic device is aligned properly with the first and second components in accordance with some embodiments;

FIG. 2A is a first side view of another example of first and second components each having a respective alignment member in accordance with some embodiments;

FIG. 2B is a representation of the first and second components illustrated in FIG. 2A being viewed under fluoroscopy when there is a misalignment between the fluoroscopic device and the first and second components in accordance with some embodiments;

FIG. 2C is a representation of the first and second components illustrated in FIG. 2A being viewed under fluoroscopy when the fluoroscopic device is aligned properly with the first and second components in accordance with some embodiments;

FIG. 3A is a first side view of another example of first and second components each having a respective alignment member in accordance with some embodiments;

FIG. 3B is a representation of the first and second components illustrated in FIG. 3A being viewed under fluoroscopy when there is a misalignment between the fluoroscopic device and the first and second components in accordance with some embodiments;

FIG. 3C is a representation of the first and second components illustrated in FIG. 3A being viewed under fluoroscopy when the fluoroscopic device is aligned properly with the first and second components in accordance with some embodiments;

FIG. 4 is a side view of one example of an alignment adapter in accordance with some embodiments;

FIG. 5A is a first side view of an alignment device in accordance with some embodiments;

FIG. 5B is a cross-sectional view of the alignment device illustrated in FIG. 5A taken along line 5B-5B in FIG. 5A positioned beneath a fluoroscopic device in accordance with some embodiments;

FIG. 5C is a cross-sectional view of the alignment device illustrated in FIG. 5A taken along line 5C-5C in FIG. 5A positioned beneath a fluoroscopic device in accordance with some embodiments; and

FIG. 5D is a cross-sectional view of the alignment device illustrated in FIG. 5A taken along line 5C-5C in FIG. 5A in use with another system in accordance with some embodiments.

### DETAILED DESCRIPTION

This description of the exemplary embodiments is intended to be read in connection with the accompanying drawings, which are to be considered part of the entire written description. The drawing figures are not necessarily to scale, and certain features may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. When only a single machine is illustrated, the term “machine” shall also be taken to include any collection of machines that individually or jointly execute a set (or multiple sets) of instructions to perform any one or more of the methodologies discussed herein. The term “operatively connected” is such an attachment, coupling or connection that allows the pertinent structures to operate as intended by virtue of that relationship. In the claims, means-plus-function clauses, if used, are intended to cover the structures described, suggested, or rendered obvious by the written description or drawings for performing the recited function, including not only structural equivalents but also equivalent structures.

Fluoroscopy is commonly used during surgical procedures to ensure the proper alignment and/or orientation of prosthesis, fixture, and/or jig relative to a patient's anatomy. However, care must be taken to ensure that the fluoroscopic device (e.g., x-ray, C-arm) is properly aligned with the prosthesis, fixture, and/or jig.

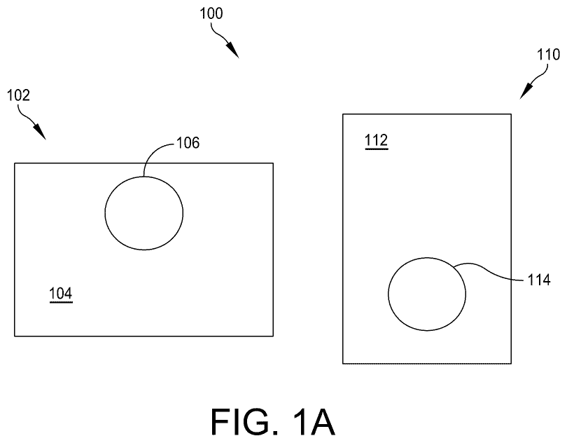

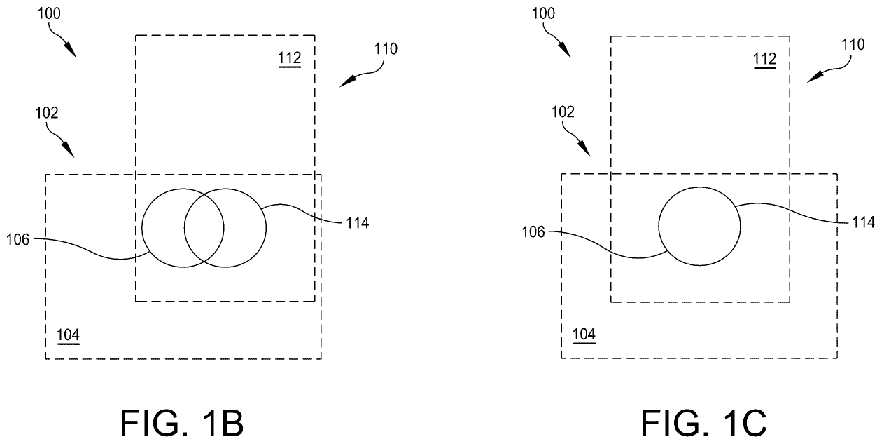

FIG. 1A illustrates one example of a system 100 in accordance with some embodiments. In some embodiments, system 100 includes a first component 102 having a body 104 supporting a first alignment member 106. A second component 110 has a body 112 supporting a second alignment member 114. The bodies 104, 112 of the first and second components 102, 110 may be formed from a radiolucent material, including but not limited to polymers, PEEK, ABS, Nylon, etc., such that the bodies 104, 112 are invisible or only partially visible when viewed under fluoroscopy as will be understood by one of ordinary skill in the art. Although first and second components 102, 110 are shown as being separate components, it should be understood that the first and second components may be configured as first and second portions of a common device (e.g., the same prosthesis, fixture, and/or jig), but disposed at a distance from one another.

Further, although first and second components 102, 110 are described as each including a single alignment member, or parallax cues, i.e., alignment members 106, 114, first and second components 102, 110 may include plural alignment members, which may be oriented in the same or different planes. For example, first and second components 102, 110 may include one or more alignment members 106, 114 disposed in a first plane (e.g., coronal plane) and may also include one or more alignment members disposed in a second plane (e.g., sagittal or transverse plane). The alignment members may communicate position in 3D space while accounting for 2D fluoroscopy and inherent parallax in the image processing.

In some embodiments, the first and second alignment members 106, 114 are formed from a radiopaque material such that the first and second alignment members may be visualized under fluoroscopy. As seen in FIG. 1A, the first and second alignment members 106, 114 have the same shape and dimensions as one another for reasons discussed herein. Although the first and second alignment members 106, 114 are shown as having a circular or ring shape, it should be understood that the shape and size of the alignment members 106, 114 may vary. Further, the radiodensity (or radiopacity) of alignment members 106, 114 may be the same or different. Radiopaque materials may include metals, alloys, or other dense elements, such as titanium or tantalum.

The manner in which the first and second alignment members 106, 114 are supported by the first and second components 102, 110 also may vary. For example, the alignment members 106, 114 may be embedded within the respective bodies 104, 112 of the first and second components 102, 110. In some embodiments, the alignment members 106, 114 are affixed to a surface of the first and second components 102, 110, such as by using an adhesive or mechanical coupling. It should be understood that the first and second alignment members 106, 114 may be supported by the first and second components 102, 110 through different means. For example, the first alignment member 106 may be embedded within body 104 of first component 102, and second alignment member 114 may be affixed to a surface of body 112 of second component 110.

FIG. 1B illustrates one example of the system 100 being viewed under fluoroscopy when there is a misalignment between the fluoroscopic device (e.g., X-ray and/or C-arm) and an anatomical plane, and FIG. 1C illustrates one example of the system 100 being view under fluoroscopy when the fluoroscopic device (e.g., X-ray and/or C-arm) are aligned properly with an anatomical plane. As is apparent through a comparison of FIGS. 1B and 1C, the misalignment is identifiable when both alignment members 106, 114 are visible under fluoroscopy (FIG. 1B) and the fluoroscopic device is properly aligned with the system when the two alignment members 106, 114 appear as one (FIG. 1C).

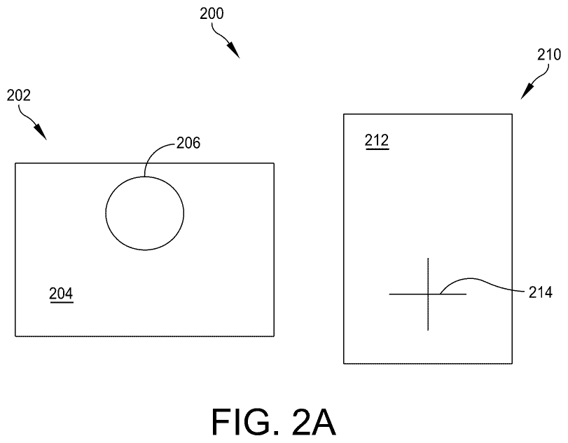

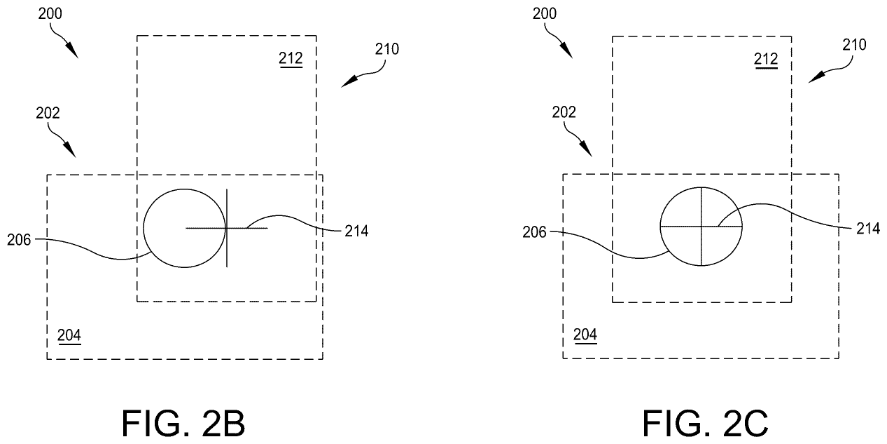

FIG. 2A illustrates another example of a system in accordance with some embodiments. System 200 includes a first component 202 having a body 204 supporting a first alignment member 206. A second component 210 has a body 212 supporting a second alignment member 214. The bodies 204, 212 of the first and second components 202, 210 may be formed from a radiolucent material such that the bodies 204, 212 are invisible or only partially visible when viewed under fluoroscopy as will be understood by one of ordinary skill in the art. Although first and second components 202, 210 are shown as being separate components, it should be understood that the first and second components may be configured as first and second portions of a common device (e.g., the same prosthesis, fixture, and/or jig), but disposed at a distance from one another.

In some embodiments, the first and second alignment members 206, 214 are formed from a radiopaque material such that the first and second alignment members may be visualized under fluoroscopy. As seen in FIG. 2A, the first and second alignment members 206, 214 have different complementary shapes. More particularly, alignment member 206 is shown as being in the shape of a circle and alignment member 214 is shown as being in the shape of a crosshair that is dimensioned to correspond to the shape of alignment member 206. It should be understood that although alignment member 206 is illustrated as being a circle and alignment member 214 is illustrated as a crosshair, alignment members 206, 214 may have other shapes or configurations. For example, alignment member 206 may have another shape, such as a square, smaller ring, rectangle, triangle, etc., and/or alignment member 214 may take the form of a dot, circle, or other shape that is designed to provide an indication of alignment as will be understood by one of ordinary skill in the art. Further, the radiodensity (or radiopacity) of alignment members 206, 214 may be the same or different.

Although first and second components 202, 210 are described as each including a single alignment member, i.e., alignment members 206, 214, first and second components 202, 210 may include plural alignment members, which may be oriented in the same or different planes. For example, first and second components 202, 210 may include one or more alignment members 206, 214 disposed in a first plane (e.g., coronal plane) and may also include one or more alignment members disposed in a second plane (e.g., sagittal or transverse plane).

The manner in which the first and second alignment members 206, 214 are supported by the first and second components 202, 210 may also vary. For example, the alignment members 206, 214 may be embedded within the respective bodies 204, 212 of the first and second components 202, 210. In some embodiments, the alignment members 206, 214 are affixed to a surface of the first and second components 202, 210, such as by using an adhesive or mechanical coupling. It should be understood that the first and second alignment members 206, 214 may be supported by the first and second components 202, 210 through different means. For example, the first alignment member 206 may be embedded within body 204 of first component 202, and second alignment member 214 may be affixed to a surface of body 212 of second component 210.

FIG. 2B illustrates one example of the system 200 being viewed under fluoroscopy when there is a misalignment between the fluoroscopic device (e.g., X-ray and/or C-arm) and an anatomic plane, and FIG. 2C illustrates one example of the system 200 being view under fluoroscopy when the fluoroscopic device (e.g., X-ray, imaging device, and/or C-arm) is aligned properly with an anatomical plane. As is apparent through a comparison of FIGS. 2B and 2C, the misalignment is identifiable when alignment member 214 is not positioned properly with respect to alignment member 206, i.e., crosshair is not disposed within and encircled by the circle (FIG. 2B), and the fluoroscopic device is aligned properly with system 200 when alignment member 214 is disposed within and encircled by alignment member 206 (FIG. 2C).

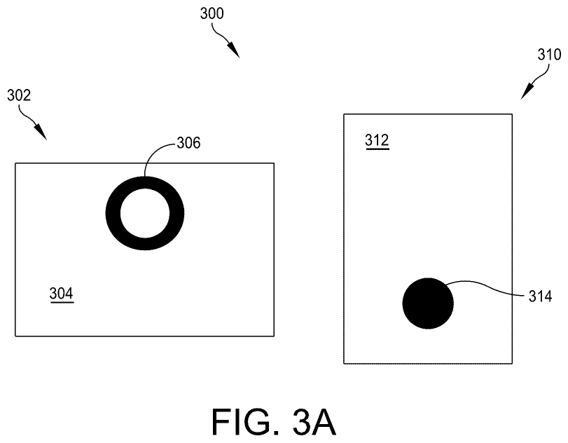

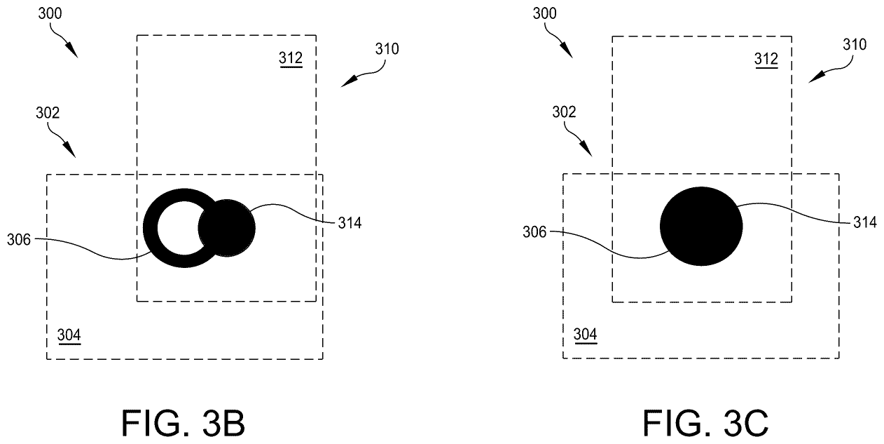

FIG. 3A illustrates another example of a system in accordance with some embodiments. System 300 includes a first component 302 having a body 304 supporting a first alignment member 306. A second component 310 has a body 312 supporting a second alignment member 314. The bodies 304, 312 of the first and second components 302, 310 may be formed from a radiolucent material such that the bodies 304, 312 are invisible or only partially visible when viewed under fluoroscopy as will be understood by one of ordinary skill in the art. Although first and second components 302, 310 are shown as being separate components, it should be understood that the first and second components may be configured as first and second portions of a common device (e.g., the same prosthesis, fixture, and/or jig), but disposed at a distance from one another.

In some embodiments, the first and second alignment members 306, 314 are formed from a radiopaque material such that the first and second alignment members may be visualized under fluoroscopy. As seen in FIG. 3A, the first and second alignment members 306, 314 have different complementary shapes. More particularly, alignment member 306 is shown as being in the shape of a circle and alignment member 314 is shown as being a dot that is sized and configured to fill the entirety of the circle of alignment member 306. In some embodiments, the radiodensity (or radiopacity) of alignment members 306, 314 may be the same or different.

Further, although first and second components 302, 310 are described as each including a single alignment member, i.e., alignment members 306, 314, first and second components 302, 310 may include plural alignment members, which may be oriented in the same or different planes. For example, first and second components 302, 310 may include one or more alignment members 306, 314 disposed in a first plane (e.g., coronal plane) and may also include one or more alignment members disposed in a second plane (e.g., sagittal or transverse plane).

The manner in which the first and second alignment members 306, 314 are supported by the first and second components 302, 310 also may vary. For example, the alignment members 306, 314 may be embedded within the respective bodies 304, 312 of the first and second components 302, 210. In some embodiments, the alignment members 306, 314 are affixed to a surface of the first and second components 302, 310, such as by using an adhesive or mechanical coupling. It should be understood that the first and second alignment members 306, 314 may be supported by the first and second components 302, 310 through different means. For example, the first alignment member 306 may be embedded within body 304 of first component 302, and second alignment member 314 may be affixed to a surface of body 312 of second component 310.

FIG. 3B illustrates one example of the system 300 being viewed under fluoroscopy when there is a misalignment between the fluoroscopic device (e.g., X-ray and/or C-arm) and an anatomical plane, and FIG. 3C illustrates one example of the system 300 being view under fluoroscopy when the fluoroscopic device (e.g., X-ray, imaging device, and/or C-arm) aligned properly with an anatomical plane. As is apparent through a comparison of FIGS. 3B and 3C, the misalignment is identifiable when alignment member 314 is not positioned properly with respect to alignment member 306, i.e., dot is not disposed within and fill the circle (FIG. 3B), and the fluoroscopic device is aligned properly with system 300 when alignment member 314 is disposed within and fills alignment member 306 (FIG. 3C).

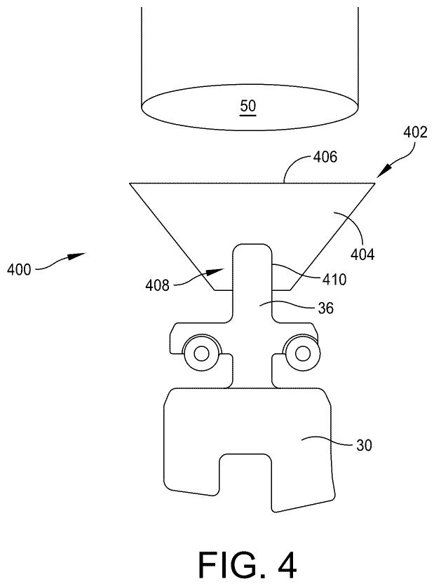

FIG. 4 illustrates another example of a system in accordance with some embodiments. More particularly, the alignment system provided in FIG. 4 includes an alignment adapter 402 that is sized and configured to engage another surgical device 30, such as the alignment guide 30 disclosed in U.S. Pat. No. 9,402,640, entitled “Alignment Guide with Embedded Features for Intra-Operative Fluoro-Checks,” which is incorporated by reference herein in its entirety.

Adapter 402 includes a body 404 that includes an enlarged planar surface 406 and a coupling mechanism 408. In some embodiments, planar surface 406 is enlarged with respect to the rest of the body 404 and provides for enhanced visualization such that the alignment between the surgical device 30 and the fluoroscopic device 50 may be checked or confirmed as described below. Coupling mechanism 408 may take a variety of forms to facilitate the coupling of adapter 402 to surgical device 30. For example and as depicted in FIG. 4, coupling mechanism 408 may the take the form of a channel 410 that is sized and configured to receive and/or engage at least a portion (e.g., the anterior protruding portion 36) of surgical device 30. It should be understood that the size and nature of coupling mechanism 408 may vary based on the characteristics of the device 30. Examples of other coupling mechanisms include, but are not limited to, a slot with dovetail connection, a detent, a hole for receiving a pin, and a protrusion designed to be inserted into a slot or hole defined by device 30, to list only a few possible examples.

Coupling mechanism 408 is designed to engage device 30 such that planar surface 406 is oriented relative to device 30 in a predetermined manner. For example, in some embodiments, coupling mechanism 408 is sized and configured to engage device 30 such that planar surface is parallel to a longitudinal axis of a radiopaque member (e.g., radiopaque members 34a, 34b, 35 disclosed in U.S. Pat. No. 9,402,640) supported by device 30.

With adapter 402 coupled to device 30, a surgeon, radiographic technician, or other individual may align the fluoroscopic device 50 with device 30 by visually determining whether the planar surface 406 of adapter 302 is oriented parallel to fluoroscopic device 50. The enlarged surface 406 provided by adapter 402 is easier to align with fluoroscopic device 50 compared to a surface feature of device 30. In some embodiments, adapter 402 and surgical device 30 may include cooperative alignment members, such as the alignment members described above with reference to FIGS. 1A-1C, 2A-2C, and 3A-3C.

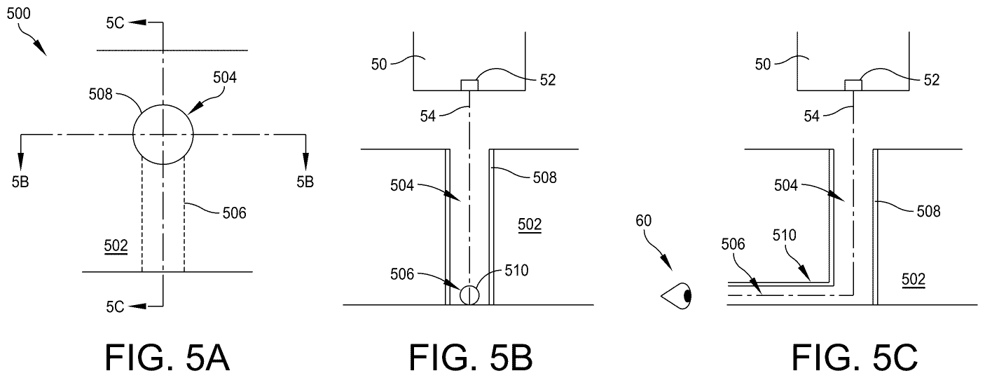

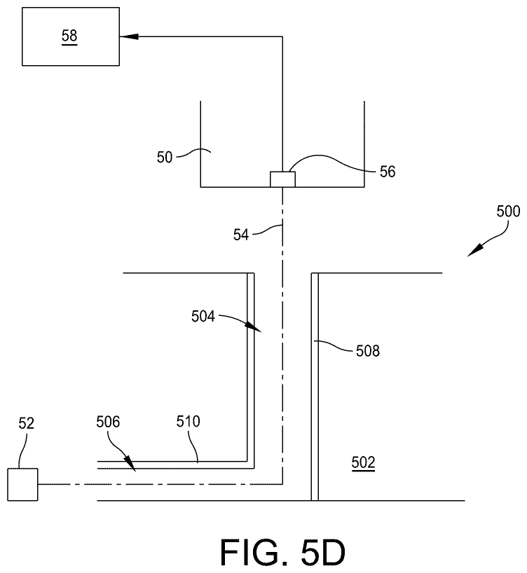

FIGS. 5A-5C illustrates another example of an alignment device 500 in accordance with some embodiments. More particularly, FIG. 5A is a top side view of one example of an alignment device 500, FIG. 5B is a cross-sectional view of the device 500 taken along line 5B-5B in FIG. 5A, and FIG. 5C is a cross-sectional view of the device 500 taken along line 5C-5C in FIG. A.

Alignment device 500 has a body 502 that may be formed from a radiolucent material. In some embodiments, body 502 defines first and second apertures 504, 506 that are oriented at an angle with respect to one another. For example, apertures 504, 506 may be orthogonally oriented relative to one another such that aperture 504 is disposed parallel to a first plane (e.g., coronal, medial, sagittal, or transverse plane) and aperture 506 is disposed parallel to a second plane that is different from the first plane (e.g., another of the coronal, medial, sagittal, and transverse planes). However, it should be understood that apertures 504, 506 may be disposed at other angles (e.g., oblique or obtuse angles) relative to one another. As best seen in FIGS. 5B and 5C, apertures 504, 506 intersect one another within body 502.

In some embodiments, liners 508, 510 are disposed within apertures 504, 506, respectively. Liners 506, 508 may be formed from a reflective material and be configured to direct light 54, which is provided by a laser, light emitting diode (LED), or illuminating device 52, along their lengths. In some embodiments, the illuminating device 52 is supported by or coupled to an fluoroscopic device 50, which may be supported by or include a C-arm as will be understood by one of ordinary skill in the art.

In use, alignment device 500 is positioned either one a patient or coupled to another surgical instrument, such as a prosthetic, fixture, and/or jig. An illuminating device or light source 52, which is coupled to or provided by a C-arm and/or fluoroscopic device 50, directs light 54 into one of the apertures (e.g., aperture 504 as shown in FIGS. 5A-5C). When the fluoroscopic device is properly aligned with the device 500, the light 54 is received within aperture 504 (and liner 508, if provided) and is visible to the surgeon via aperture 506 (and liner 510, if provided). As noted above, the liners 508, 510 may reflect the light along their lengths to enable the light to be viewed by the surgeon, technician, or other individual 60 (FIG. 5C) and prevents the device 500 from being illuminated entirely by illuminating device 52.

FIG. 5D illustrates another system in which alignment device 500 may be included. As shown in FIG. 5D, a light source 52 directs light into aperture 506 (and liner 510, if provided) and a camera or optical sensor 56 is provided on the fluoroscopic device 50 (e.g., X-ray lens, C-arm, etc.) to detect light exiting aperture 504 (guided by liner 508, if provided). In some embodiments, the camera 56 is coupled to a control and image processing system 58, which may be configured to detect the light emanating from aperture 504 and move the C-arm and fluoroscopic device into position based on the light detected by camera or optical sensor 56. One example of a control and image processing system is imaging system 200 disclosed in U.S. Pat. No. 8,611,697, which is incorporated by reference herein in its entirety.

In some embodiments, a system includes a first component and a second component. The first component has a first body supporting a first alignment member. The second component has a second body supporting a second alignment member. The first and second alignment members are separated from another and are configured to provide an indication that a fluoroscopic device is properly aligned with an anatomical plane when viewed under fluoroscopy.

In some embodiments, the first component and the second component are respective portions of a common device.

In some embodiments, a shape of the first alignment member is the same as a shape of the second alignment member.

In some embodiments, a dimension of the first alignment member is the same as a dimension of the second alignment member.

In some embodiments, a radiopacity of the first alignment member is different from a radiopacity of the second alignment member.

In some embodiments, a shape of the first alignment member is different from a shape of the second alignment member.

In some embodiments, the shape of the first alignment member is complementary to the shape of the second alignment member.

In some embodiments, the shape of the first alignment member is circular.

In some embodiments, the second alignment member includes a crosshair.

In some embodiments, the shape of the second alignment member is a dot sized to fill an opening of the circular shape of the first alignment member.

In some embodiments, a method includes placing a first component supporting a first alignment member and a second component supporting a second alignment member relative to a patent, and aligning a fluoroscopic device with an anatomical plane using the first and second alignment members.

In some embodiments, aligning the fluoroscopic device with the anatomical plane includes viewing the first and second alignment members under fluoroscopy.

In some embodiments, the first and second alignment members appear as a single alignment member when the fluoroscopic device is aligned with the anatomical plane.

In some embodiments, the first and second alignment members have a complementary shape when the fluoroscopic device is aligned with the anatomical plane.

In some embodiments, a system includes an alignment device having a body defining a first aperture and a second aperture. The first and second apertures are disposed at an angle with one another. The first and second apertures are in communication with one another such that light passes through the first and second apertures when a fluoroscopic device is aligned with an anatomical plane.

In some embodiments, a first liner is disposed within the first aperture. The first liner is configured to direct light along a length of the first aperture.

In some embodiments, a second liner is disposed within the second aperture. The second liner is configured to direct light along a length of the second aperture.

In some embodiments, a system includes a light source configured to direct light into at least one of the first aperture and the second aperture.

In some embodiments, the light source is coupled to a C-arm of a radiographic device.

In some embodiments, a system includes an optical sensor coupled to a C-arm of a radiographic device. The optical detector is configured to detect light exiting at least one of the first aperture and the second aperture.

In some embodiments, a system includes a camera coupled to a C-arm of a radiographic device. The camera is configured to receive light from at least one of the first aperture and the second aperture.

In some embodiments, a method includes receiving light emitted from a light source in a first aperture defined by an alignment device, and directing the light from the first aperture defined by the alignment device to a second aperture defined by the alignment device such that light exits the alignment device from the second aperture. The second aperture is disposed at an angle with respect to the first aperture. The second aperture is disposed at an angle with respect to the first aperture.

In some embodiments, the light emitted from the light source is received within the first aperture when a fluoroscopic device is aligned with an anatomical axis.

In some embodiments, a method includes detecting light exiting the alignment device using an optical sensor.

In some embodiments, a method includes adjusting a position of a fluoroscopic device if light is not detected by the optical sensor.

In some embodiments, a method includes detecting light exiting the alignment device using a camera.

In some embodiments, a method includes adjusting a position of a fluoroscopic device if light is not detected by the camera.

In some embodiments, a system includes a first surgical device and an alignment adapted. The first surgical device is configured to be coupled to a patient. The alignment adapter has a body including a planar surface and a coupling mechanism for coupling the alignment adapter to the first surgical device such that the planar surface is parallel to an anatomical plane when the first surgical device is coupled to the patient and the alignment adapter is coupled to the first surgical device.

In some embodiments, the coupling mechanism includes a channel sized and configured to receive at least a portion of the first surgical device.

In some embodiments, the coupling mechanism includes a detent sized and configured to engage the first surgical device.

In some embodiments, the coupling mechanism includes a slot having a dovetail that is sized and configured to engage a corresponding feature of the first surgical device.

In some embodiments, a method includes placing a first surgical device relative to a patient, coupling an alignment device to the first surgical device, and aligning a fluoroscopic device with the planar surface of the alignment device. The alignment device includes a planar surface.

In some embodiments, a method includes adjusting a position of the fluoroscopic device until the fluoroscopic device is aligned with the planar surface of the alignment device.

In some embodiments, the planar surface of the alignment device is parallel to an anatomical plane of the patient.

Although the systems and methods have been described in terms of exemplary embodiments, they are not limited thereto. Rather, the appended claims should be construed broadly, to include other variants and embodiments of the systems and methods, which may be made by those skilled in the art without departing from the scope and range of equivalents.

What is claimed is:

1. A method, comprising:
placing a first component supporting a first alignment member in a first plane and a second component supporting a second alignment member in a second plane and relative to a patient; and
aligning a fluoroscopic device with an anatomical plane using the first and second alignment members to thereby locate and communicate a position in 3D space when viewed under fluoroscopy so that the first and second alignment members appear as a single alignment member when the fluoroscopic device is aligned with the anatomical plane.

2. The method of claim 1, wherein aligning the fluoroscopic device with the anatomical plane includes viewing the first and second alignment members under fluoroscopy.

3. The method of claim 2, wherein the first and second alignment members have a complementary shape when the fluoroscopic device is aligned with the anatomical plane.

4. A system comprising:
a first component having a first body defining a first aperture and supporting two first alignment members; and
a second component having a second body defining a second aperture and supporting two second alignment members, wherein a first one of the two first alignment members and a first one of the two second alignment members are disposed in a first plane, and a second one of the two first alignment members and a second one of the two second alignment members are disposed in a second plane, wherein each of the two first alignment members are separated from each of the two second alignment members such that the two first alignment members and the two second alignment members are configured so that the first and second apertures are in communication with one another such that light passes through the first and second apertures when a fluoroscopic device is aligned with an anatomical plane to indicate alignment and to communicate a position in 3D space.

5. The system of claim 4, wherein a first liner is disposed within the first aperture, the first liner configured to direct light along a length of the first aperture.

6. The system of claim 5, wherein a second liner is disposed within the second aperture, the second liner configured to direct light along a length of the second aperture.

7. The system of claim 6, further comprising a light source configured to direct light into at least one of the first aperture and the second aperture.

8. The system of claim 7, wherein the light source is coupled to a C-arm of a radiographic device.

9. The system of claim 8, further comprising an optical sensor coupled to a C-arm of a radiographic, the optical detector configured to detect light exiting at least one of the first aperture and the second aperture.

10. The system of claim 8, further comprising a camera coupled to a C-arm of a radiographic device, the camera configured to receive light from at least one of the first aperture and the second aperture.

11. A method, comprising:
receiving light emitted from a light source in a first aperture defined by an alignment device when a fluoroscopic device is aligned with an anatomical axis; and
directing the light from the first aperture defined by the alignment device to a second aperture defined by the alignment device such that light exits the alignment device from the second aperture, the second aperture disposed at an angle with respect to the first aperture,
wherein the second aperture is disposed at an angle with respect to the first aperture.

12. The method of claim 11, further comprising detecting light exiting the alignment device using an optical sensor.

13. The method of claim 11, further comprising adjusting a position of a fluoroscopic device if light is not detected by the optical sensor.

14. The method of claim 11, further comprising detecting light exiting the alignment device using a camera.

15. The method of claim 11, further comprising adjusting a position of a fluoroscopic device if light is not detected by the camera.

16. A system, comprising:
a first surgical device configured to be coupled to a patient; and
an alignment adapter having a body, the body including:
a planar surface, and
a coupling mechanism including a channel sized and configured to receive at least a portion of the first surgical device for coupling the alignment adapter to the first surgical device such that the planar surface is parallel to an anatomical plane when the first surgical device is coupled to the patient and the alignment adapter is coupled to the first surgical device.

17. The system of claim 16, wherein the coupling mechanism includes a detent sized and configured to engage the first surgical device.

18. The system of claim 16, wherein the coupling mechanism includes a slot having a dovetail that is sized and configured to engage a corresponding feature of the first surgical device.

19. The system of claim 18, wherein the light source is coupled to a C-arm of a radiographic device.

20. The system of claim 18, further comprising an optical sensor coupled to a C-arm of a radiographic device, the optical detector configured to detect light exiting at least one of the first aperture and the second aperture.

21. The system of claim 18, further comprising a camera coupled to a C-arm of a radiographic device, the camera configured to receive light from at least one of the first aperture and the second aperture.

22. A method, comprising:
receiving light emitted from a light source in a first aperture defined by an alignment device when a fluoroscopic device is aligned with an anatomical axis; and
directing the light from the first aperture defined by the alignment device to a second aperture defined by the alignment device such that light exits the alignment device from the second aperture, the second aperture disposed at an angle with respect to the first aperture, wherein the second aperture is disposed at an angle with respect to the first aperture; and
detecting light exiting the alignment device using an optical sensor.

23. The method of claim 22, further comprising adjusting a position of a fluoroscopic device if light is not detected by the optical sensor.

24. The method of claim 22, further comprising detecting light exiting the alignment device using a camera.

25. The method of claim 24, further comprising adjusting a position of a fluoroscopic device if light is not detected by the camera.
